(19) United States
(12) Patent Application Publication
(10) Pub. No.: US 2003/0044354 A1
(43) Pub. Date: Mar. 6, 2003

(54) Gas microsphere liposome composites for ultrasound imaging and ultrasound stimulated drug release

(76) Inventors: Alan P. Carpenter JR., Carlisle, MA (US); Gregory C. Slack, Hollis, NH (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 09/931,317

(22) Filed: Aug. 16, 2001

(51) Int. Cl. A61B 8/00

(52) U.S. Cl. 424/9.51

(57) ABSTRACT

Formulations comprising a gas microsphere liposome composite suspended in a medium, wherein the gas microsphere liposome composite comprises: a gas-filled microsphere; at least one of a lipid and a surfactant adsorbed onto the surface of the gas-filled microsphere; and liquid-filled liposomes attached to the lipid or surfactant are described. Methods of preparing the same and using them in ultrasound imaging are also described. The present invention also comprises use of the same in treating heart disease, inflammation, infection, cancer or thromboembolic disease in a patient

### FIELD OF THE INVENTION

The present invention provides a formulation that includes a gas microsphere liposome composite (MSLC) suspended in a medium. The gas microsphere liposome composite includes a gas-filled microsphere; at least one of a lipid and a surfactant adsorbed onto the surface of the gas-filled microsphere; and liquid-filled liposomes attached to the lipid or surfactant. The outer surface of the liquid-filled liposomes can incorporate a targeting ligand (i.e., diagnostic agent targeting moiety) for directed delivery of the MSLCs for selective imaging of receptors, enzymes, MRNA and other relevant biological targets. Additionally, the liquid-filled liposomes can include one or more drugs (e.g., therapeutic agents and/or diagnostic agents) in the internal volume of the liquid-filled liposomes. As such, the therapeutic agent or diagnostic agent can be selectively delivered to an organ or site of pathology for localized delivery. Accelerated drug release can be stimulated by the application of acoustic energy at the site of pathology where the targeted MSLCs bind, thereby providing locally high concentrations of therapeutic agent or diagnostic agent in a selective fashion.

### BACKGROUND OF THE INVENTION

Ultrasound imaging is useful for imaging structures in the body of a patient (e.g., mammal) so as to aid in diagnosis and therapy. During ultrasound imaging, an ultrasonic scanner can be used to generate and receive sound waves. The ultrasonic scanner is placed on the body surface overlying the area to be imaged, and the sound waves generated by the scanner are directed toward the area to be imaged. The scanner then detects sound waves reflected from the underlying area and translates the data into images. The acoustic properties (e.g., density) of each structure in the body will typically depend upon the velocity of the transmissions and the density of the structure. Changes in acoustic properties will be most prominent at the interface between different substances (i.e., at the interface between solids, liquids and gases). Thus, when ultrasonic energy is directed at an area that includes interfaces between different substances, the different acoustic properties of the substances will cause different reflection characteristics. Because the quality of the resulting ultrasound image is enhanced by having an interface between different structures, it would be useful to increase the difference in acoustic properties between different structures and to enhance the quality of the image generated during ultrasound imaging.

One method that can affect the quality of ultrasound imaging is the introduction of contrast agents into the vasculature of the body to act as ultrasound contrast agents. When the contrast agents are injected into and perfuse the microvasculature, clearer images may be produced. The agents act as sound wave reflectors, effectively enhancing the interface between the vasculature and other structures.

Liquid and solid contrast agents containing entrapped gas are well known in the art. See, e.g., U.S. Pat. Nos. 4,235,871; 4,265,251; 4,442,843; 4,533,254; 4,572,203; 4,657,756; 4,681,199; 5,088,499; 5,147,631; 5,228,446; 5,271,928; 5,380,519; 5,413,774; 5,527,521; 5,531,980; 5,547,656; 5,558,094; 5,573,751; 5,585,112; 5,620,689; 5,715,824; 5,769,080; EP 0 122 624; EP 0 727 225; WO 96/40285; and WO 99/65467. The microbubbles provided by these contrast agents act as sound wave reflectors due to the acoustic differences between the gas microbubble and surrounding liquid.

Feinstein, U.S. Pat. No. 4,572,203, discloses “microbubbles” of about 6-20 microns in diameter, produced by sonication of certain viscous solutions, for use as ultrasound contrast agents. Feinstein also discloses solid or semi-solid metal-containing microparticles, such as glass or graphite, not containing trapped air, small enough to pass through capillaries, as ultrasound contrast agents. Also disclosed are microspheres formed from an amino acid polymer matrix, such as albumin, with magnetic particles, such as magnetite (Fe₃O₄) embedded therein.

Tickner, U.S. Pat. No. 4,265,251, discloses the use of certain saccharide composition “microbubble” particles with a hollow gas-filled interior space as ultrasound enhancing agents.

Rasor et al., U.S. Pat. Nos. 4,442,843, 4,657,756, and 4,681,119, illustrate aggregates of microparticles (of 1-50 micron diameter) of a solid material, which are soluble in blood, containing gas in the voids between the particles, or with gas adsorbed on the surface of the particle, or containing gas as an integral part of the internal structure of the particle, for use in ultrasound imaging. The following solid materials are used: various saccharides, NaCl, sodium citrate, sodium acetate, sodium tartrate, CaCl₂ and AlCl₃.

Hilmann et al., EP0122624, contains microparticles that include a solid surface-active substance, including various organic lipophilic compounds, with enclosed air, for use as ultrasound contrast agents. Also disclosed is the combination of particles of the surface-active material and particles of a non-surface active material, such as sodium chloride, sodium citrate, sodium acetate, sodium tartrate, and various saccharides.

Glajch et al, U.S. Pat. No. 5,147,631, discloses porous particles of an inorganic material that include an entrapped gas or liquid. The materials disclosed include monomeric or polymeric borates, monomeric or polymeric aluminas, monomeric or polymeric carbonates, monomeric or polymeric silicas, monomeric or polymeric phosphates; and pharmaceutically acceptable organic or inorganic cationic salts thereof.

Unger disclosed perfluorocarbon gas-filled microspheres (U.S. Pat. Nos. 5,547,656 and 5,527,521) for diagnostic imaging purposes and gas-filled and gaseous-precursor-filled liposome compositions, or methods for making or using these contrast agents (U.S. Pat. Nos. 5,228,446, 5,585,112, 5,769,080 and 5,715,824)) for general and diagnostic ultrasound imaging purposes.

Unger, U.S. Pat. No. 5,088,499, discloses the preparation of gas filled liposomes and their use as ultrasound contrast agents. These include materials that contain gases, gaseous precursors, which can be activated by pH, temperature, or pressure, and other solid and liquid contrast agents.

In the case of the materials disclosed by Unger herein above, the liposomal membrane of the encapsulated gas bubble is described as the well-known unilamellar or multi-lamellar head-to-tail structure of amphiphilic lipid membranes such as phospholipids (see, FIG. 1). As such, the Unger compositions are classical liposomes in which the liquid-filled interior is replaced by a gas.

Quay has disclosed methods of use of free gas microbubbles of low Q-factor (low diffusivity) as ultrasound contrast agents (U.S. Pat. Nos. 5,573,751 and 5,558,094). In these cases Quay discloses free gas microbubbles of various low diffusivity gases, without any disclosure of structure or composition of these microbubbles.

Schneider, U.S. Pat. Nos. 5,271,928, 5,380,519 and 5,531,980, disclosed microbubble suspensions, which are hollow spheres or globules of finely divided gas and are stabilized by tensides or surfactants.

In the case of Schneider microbubble patents ('928,'519 and '980), the ultrasound contrast agent is disclosed as being composed of microbubbles devoid of a material boundary layer around the gas microbubble. According to Schneider, these microbubbles “are only bounded by an evanescent envelope” (U.S. Pat. No. 5,531,980, column 1).

The Schneider microbubble disclosures described above ('928,'519 and '980) are directed to methods of making microbubble-based ultrasound contrast agents without reference to preferred composition/structure of the microbubbles themselves.

Schneider, U.S. Pat. No. 5,413,774, discloses microvesicles having a liposomal material boundary layer, which further contain within the vesicle a low solubility gas, as the microsphere-based ultrasound contrast agents. However, no description of the composition or structure of the microballoons is provided; rather, methods of making a contrast agent based on these microvesicles or microballoons is described utilizing selected low solubility gases.

The contrast agents described above are proposed for general ultrasound contrast imaging of the vasculature and especially for heart imaging.

The imaging of specific organs, systems, or other areas of the body, would be useful for diagnosing a variety of specific disease states. Examples of this include the specific imaging of tumors, blood clots, and areas of infection in a directed manner. Quay, et al., European Patent Application EP727225 illustrates the use of compositions including a cell adhesion molecule (CAM) ligand which is incorporated into a desired molecule to form a conjugate. The CAM is incorporated in a surfactant or albumin carrier and also comprises a chemical with sufficiently high vapor pressure to be a gas at body temperature.

Unger (WO 96/40285) describes targeted gas-containing liposomes which can be targeted to specific tissues in the body for diagnostic imaging or for delivery of bioactive agents. These targeted materials are comprised of a gas, lipid and targeting ligand.

All of these materials include a suspension or emulsion of gas microspheres (alternatively referred to as microbubbles) which are either: 1) free microbubbles (i.e., do not have a fixed material envelope at the microbubble surface) stabilized by surfactants in solution which cause a reduction in surface tension at the gas-liquid interface, or 2) true vesicles with a material boundary layer which stabilizes the gas microspheres as a suspension in the liquid medium. One of the practical difficulties with all of these materials is that gas microbubbles in the relevant, acoustically-active, size range of ˜0.5 μm to 10 μm in diameter, have a density different from that of the aqueous media in which they are suspended. Therefore, these microspheres have a natural tendency to rapidly separate out (i.e., the microbubble suspensions become heterogenous). This necessitates the rapid use of the contrast material after mixing before separation of the microspheres occurs.

In the case of gas microspheres used as platforms for drug delivery (see, Unger WO 96/40285 and Quay EP0727225), the materials incorporate the therapeutic moiety at the surface of the gas microsphere through chemical or physical absorption on the boundary layer of lipid or polymer. The practical difficulty with these materials is that limited quantities of the therapeutic agent may be absorbed or bound to the surface material surrounding the gas microsphere.

Allen et al. (U.S. Pat. No. 5,620,689) disclose a method of treating a neoplasm of B-cells or T-cells utilizing a liposome encapsulated chemotherapeutic agent with a biodirecting group on the surface of the liposome attached via a polyethylene glycol coating on the liposome. See et al., WO 99/65467, disclose a method of making drug filled liposomes of less than 200 nm in diameter. These disclosures are representative of a large class of similar liposome drug delivery disclosures in the literature, all of which comprise liquid-filled liposomes alone without a gas microsphere component in the form of the MSLC compositions provided herein.

Notwithstanding the use of such contrast agents described above, the ultrasound image produced, for example, of the myocardial tissue, can be of relatively poor quality, highly variable and not quantifiable. The overall diagnostic results to date have been somewhat disappointing. As such, the need still exists for improved agents useful in ultrasound imaging which will enhance the quality of ultrasound images by improving the contrast between the vascular spaces and tissues in a body. Such contrast agents should have excellent and stable acoustic response properties when in dilute aqueous suspensions. Additionally, the contrast agents should exhibit minimal microsphere flotation and separation.

There has been, and continues to be, a need for ultrasound imaging agents which enhance the quality and clarity of ultrasound images by improving the delineation of vascular space and tissues in the human body. In addition, improvements in the control of drug delivery to the sites of pathology are needed for many drugs which exhibit high toxicity to normal tissues and a resultant poor therapeutic index.

### SUMMARY OF THE INVENTION

The present invention provides a formulation for contrast enhancement of ultrasound imaging and for ultrasound (i.e., acoustically) stimulated drug release. The formulation provides stable gas microsphere (i.e., finely divided gas bubbles) suspensions with excellent and stable acoustic response properties when in dilute aqueous suspensions. The formulation can deliver a higher level of active drug per gas-filled microsphere to a given tissue, relative to known formulations, thereby achieving the intended therapeutic benefit of high local concentrations of drug or gene in the region of pathology. The formulation has good ultrasound scattering properties, which causes a selective increase in the ultrasound backscatter signal within the vascular space. The increase in the ultrasound backscatter signal within the vascular space improves the contrast relative to the surrounding solid tissue. Additionally, the formulation exhibits minimal microsphere flotation and separation.

The present invention provides a formulation that includes a gas microsphere liposome composite (MSLC) suspended in a medium. The gas microsphere liposome composite includes a gas-filled microsphere; at least one of a lipid and a surfactant adsorbed onto the surface of the gas-filled microsphere; and liquid-filled liposomes attached to the lipid or surfactant.

The present invention also provides a method of ultrasound imaging in a patient (e.g., mammal) in need of such ultrasound imaging. The method includes administering to the patient (e.g., mammal) an effective amount of a formulation of the present invention; allowing a sufficient period of time for the circulation of the gas-filled microsphere composite to reach the targeted area; and performing ultrasound imaging on the patient (e.g., mammal).

The present invention also provides a method of treating heart disease, inflammation, infection, cancer or thromboembolic disease in a patient (e.g., mammal) in need of such treatment. The method includes administering to the patient (e.g., mammal) an effective amount of a formulation of the present invention, wherein one or more of the liquid-filled liposomes independently includes a therapeutic agent; allowing a sufficient period of time for the circulation of the gas microsphere composite to the targeted area; and applying ultrasound energy to the targeted area in the patient (e.g., mammal) sufficient to cause the therapeutic agent to be released from the microsphere liposome composite at the region of pathology.

The present invention also provides a method for preparing a formulation of the present invention. The method includes contacting a suspension of liposomes in a aqueous solution including at least one of a surfactant and a lipid; and mixing the suspension with a gas that has a solubility of less than about 1.0% (v/v) in water at 25° C. and 1 atm, sufficient to provide the formulation.

The present invention also provides a method for preparing a formulation of the present invention. The method includes contacting a suspension of liposomes in a aqueous solution including at least one therapeutic agent and at least one surfactant or lipid; and mixing the aqueous liposome suspension with a gas that has a solubility of less than about 1.0% (v/v) in water at 25° C. and 1 atm, sufficient to provide the formulation.

The present invention also provides a kit for the preparation of a formulation of the present invention. The kit includes a container that includes an aqueous solution, wherein the aqueous solution includes at least one of a surfactant and a lipid, and liquid-filled liposomes; and a means for introducing a gas that has a solubility of less than about 1.0% (v/v) in water at 25° C. and 1 atm into the aqueous solution.

The present invention also provides the use of a formulation of the present invention for the manufacture of a medicament for treating heart disease, inflammation, infection, cancer or thromboembolic disease in a patient (e.g., mammal) in need of such treatment. The formulation includes a gas microsphere liposome composite suspended in a medium, wherein the gas microsphere liposome composite includes: a gas-filled microsphere; at least one of a lipid and a surfactant adsorbed onto the surface of the gas-filled microsphere; and liquid-filled liposomes attached to the lipid or surfactant.

The present invention also provides the use of a formulation of the present invention for the manufacture of a medicament for ultrasound imaging in a patient (e.g., mammal) in need of such ultrasound imaging. The formulation includes a gas microsphere liposome composite suspended in a medium, wherein the gas microsphere liposome composite includes: a gas-filled microsphere; at least one of a lipid and a surfactant adsorbed onto the surface of the gas-filled microsphere; and liquid-filled liposomes attached to the lipid or surfactant.

The present invention also provides the use of a formulation of the present invention for the manufacture of a medicament for diagnostic imaging in a patient (e.g., mammal) in need of such diagnostic imaging. The formulation includes a gas microsphere liposome composite suspended in a medium, wherein the gas microsphere liposome composite includes: a gas-filled microsphere; at least one of a lipid and a surfactant adsorbed onto the surface of the gas-filled microsphere; and liquid-filled liposomes attached to the lipid or surfactant.

### BRIEF DESCRIPTION OF THE FIGURES

FIG. 1 illustrates a gas-filled liposome.

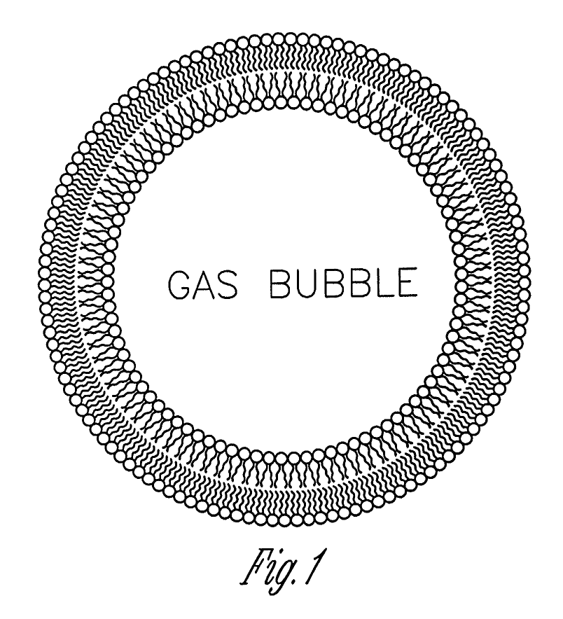

FIG. 2 illustrates a monolayer gas microsphere liposome composite (MSLC) of the present invention.

FIG. 3 illustrates a multilayer gas microsphere liposome composite (MSLC) of the present invention.

### DETAILED DESCRIPTION OF THE INVENTION

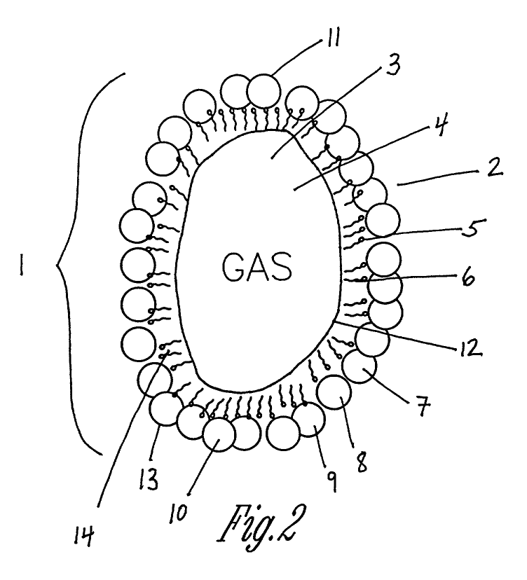

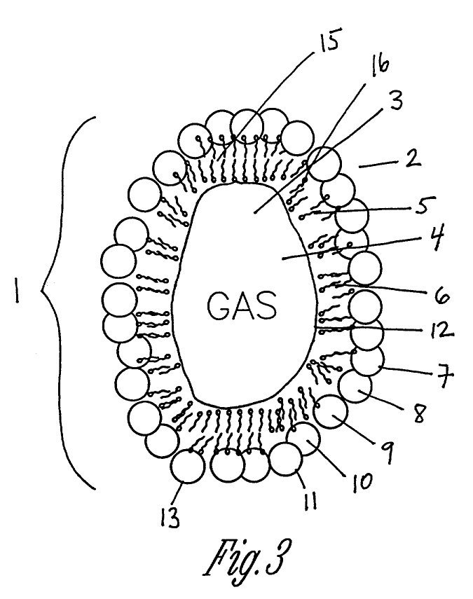

Referring to FIGS. 2 and 3, the present invention provides a gas microsphere liposome composite (MSLC) (1) dispersed in an aqueous medium (2). The gas microsphere liposome composite (MSLC) (1) includes of a gas-filled microsphere (3) of a suitable inert gas (4). A lipid (5) and/or surfactant (6) is adsorbed on the surface (12) of the gas-filled microsphere (3). Liquid-filled liposomes (LFLs) (7) are attached to the lipid (5) and/or surfactant (6). The LFLs (7) can include a therapeutic agent (8) or diagnostic agent (9) in the liquid interior (10) of the LFLs (7). In addition, a targeting moiety (11) can be attached to the surface (13) of the LFLs (7).

As used herein, “gas microsphere liposome composite (MSLC)” (1) refers to a gas-filled microsphere (3) having at least one of a lipid (5) and a surfactant (6) adsorbed onto the outer surface (12) of the gas-filled microsphere (3) and also having liquid-filled liposomes (7) attached to the lipid (5) or surfactant (6).

As used herein, “surfactant” (6) refers to any material, ionic or non-ionic, which produces a reduction in interfacial tension in a solution. The term surfactant (6) includes both amphiphilic molecules less than about 1,000 molecular weight and polymers which are capable of reducing interfacial tension between a gas-filled microsphere (3) and the surrounding aqueous medium (2).

As used herein, “liquid filled liposome (LFL)” (7) refers to liposomes that contain a liquid interior (10) (i.e., a liquid in the internal volume). The liquid filled liposomes (7) can be unilamellar (14), bilameller (15), or multilamellar (16). The liquid filled liposomes (7) are typically attached to the adsorbed liquid or surfactant (6) in a continuous fashion. Each of the liquid filled liposomes (7) can independently contain a therapeutic agent (8) or diagnostic agent (9) in the liquid interior (10) of the liquid filled liposome (7). Additionally, each of the liquid filled liposomes (7) can independently contain a high affinity, targeting moiety (11) attached to the surface (13) of the liquid filled liposome (7).

As used herein, “continuous” or “contiguous”, with respect to the liquid-filled liposomes (7) attached to the lipid (5) or surfactant (6) coated gas-filled microsphere (3) surface, refers to a significant portion (e.g., at least about 50%) of the outer surface (12) of the gas-filled microsphere (3) being covered with liquid-filled liposomes (7).

As used herein, “targeting moiety”, refers to a biocompatible organic molecule, biocompatible inorganic molecule, protein, peptide, peptidomimetic, polysaccharide or other molecule having a high affinity for a receptor, enzyme, mRNA or DNA. The biocompatible organic molecule, biocompatible inorganic molecule, protein, peptide, peptidomimetic, polysaccharide or other molecule is altered in its expression at a site of pathology in-vivo relative to the surrounding normal tissue. Additionally, this targeting moiety is principally bound or attached to the surface of the liquid-filled liposomes (7).

As used herein, “high affinity” refers to a binding affinity of less than about 1 μm when expressed as the dissociation constant, Kd, for the interaction of a single targeting moiety and the biological target (e.g., receptor, enzyme, mRNA, or DNA).

As used herein, “patient” refers to one who is suffering from a given disease or disorder and is in need of treatment for the specified disease or disorder. Suitable patients include, e.g., animals. Suitable animals include, e.g., mammals. Suitable mammals include, e.g., humans.

As used herein, “treating” or “treatment” refers to the treatment of a disease or disorder in a patient and includes: (i) preventing the disease or disorder from occurring in a patient, in particular when such patient is predisposed to the disease or disorder but has not yet been diagnosed as having it; (ii) inhibiting the disease or disorder, i.e., arresting its development; and/or (iii) relieving the disease or disorder, i.e., causing the regression of the disease or disorder.

Gas-Filled Microsphere

As used herein, a “gas-filled microsphere” is a microbubble suspended in a medium wherein the microbubble has a nominal spherical shape above about the freezing point of the medium and below about the boiling point of the medium and above about 0 atm pressure and below about 5 atm pressure (e.g., standard temperature and pressure).

As illustrated in FIG. 2 and FIG. 3, the gas microsphere liposome composite (1) (MSLC) includes a gas-filled microsphere (3). The gas-filled microsphere (3) is typically acoustically active. The gas-filled microsphere (3) typically has a solubility of less than about 1.0% (v/v) in water at 25° C. and 1 atm. Additionally, the gas-filled microsphere (3) typically has an average diameter of about 0. 1 μm to about 10 μm. Preferably, the gas-filled microsphere (3) will have an average diameter of about 0.5 μm to about 10 μm.

The gas-filled microsphere (3) will typically include one or more suitable inert gases (4). Suitable inert gases (4) of the present invention are well known in the field of ultrasound contrast agents. Suitable inert gases (4) useful in the present invention are disclosed, e.g., in Unger, et al., (U.S. Pat. Nos. 5,547,656; 5,527,521; 5,228,446; 5,585,112; 5,769,080; and 5,715,824), Quay, et al., (U.S. Pat. Nos. 5,573,751 and 5,558,094) and Schneider (U.S. Pat. Nos. 5,271,928; 5,380,519; and 5,531,980). These may include both gases and gaseous precursors (i.e., liquids which undergo a transition to the gas phase under reduced pressure or elevated temperature). Preferred inert gases (4) are of low solubility in blood, are non-reactive, non-metabolizable and/or are non-toxic in patients (e.g., mammals). Suitable inert gases (4) useful in the present invention include, e.g., perfluorocarbon gases (e.g. (C₂-C₆) perfluorocarbons), perfluoroether gases, Nitrogen, and noble gases (e.g., Helium, Argon, and Neon).

Gas Microsphere Liposome Composite (MSLC)

The gas microsphere liposome composite (1) includes a gas-filled microsphere (3); at least one of a lipid (5) and a surfactant (6) adsorbed onto the outer surface (12) of the gas-filled microsphere (3); and liquid-filled liposomes (7) attached to the lipid (5) or surfactant (6). The gas microsphere liposome composite (1) (MSLC) will typically have a mean diameter of about 0.1 μm to about 10 μm. Preferably, the gas microsphere liposome composite (1) will have a mean diameter of about 0.2 μm to about 4 μm. The gas microsphere liposome composite (1) will typically have a density of about 0.90 to about 1.10 of the density of the medium (2). The gas microsphere liposome composite (1) (MSLC) can exist as an aggregate of two or more gas microsphere liposome composites (1). The aggregate will typically have a diameter of about 1 μm to about 100 μm.

Lipid and Surfactant

As illustrated in FIG. 2 and FIG. 3, the gas microsphere liposome composite (MSLC) (1) includes at least one of a lipid (5) and surfactant (6) adsorbed onto the outer surface (12) of the gas-filled microsphere (3). The lipid (5) or surfactant (6) can exist as a mono-molecular layer, a bi-molecular layer, or a multi-molecular layer on the outer surface (12) of the gas-filled microsphere (3). The surfactant (6) rapidly adsorbs to the outer surface (12) of the gas-filled microspheres (3) and thereby reduces the surface tension of the low solubility inert gas (4) or gases. Additionally, the surfactant (6) acts as an interface to which the LFLs (7) may adhere.

The surfactant (6) can be any suitable non-ionic surfactant, cationic surfactant, or anionic surfactant. Suitable non-ionic surfactants include, e.g., polyethylene glycol, polypropylene glycol, polyvinylpyrollidone, polyvinylalcohol, cellulose, gelatin, xanthan gum, pectin, and dextran. Suitable cationic surfactants include, e.g., tetraalkyl ammonium, tetraalkyl phosphonium, or suitable salts thereof. Suitable cationic surfactants include, e.g., tetrahexyl ammonium, tetradecyl ammonium, tetrabutyl ammonium, tetrahexyl phosphonium, tetradecyl phosphonium, tetrabutyl phosphonium, tetraphenyl phosphonium, and suitable salts thereof. Suitable anionic surfactants include, e.g., alkyl sulfonate, alkyl carboxylate, and suitable salts thereof. Suitable anionic surfactants include, e.g., dodecyl sulfate, palmityl sulfate, dodecyl carboxylate, palmityl carboxylate, and suitable salts thereof.

Suitable lipids (5) include, e.g., phospholipids, glycolipids, triglycerides and fatty acids. Suitable phospholipids include, e.g., dipalmitoylphosphatidyl choline chloride, dimyristoylphosphatidyl choline, dilauryoylphosphatidyl choline, and dioleoylphosphatidyl choline.

Liquid-Filled Liposomes (LFLs)

As illustrated in FIG. 2 and FIG. 3, the gas microsphere liposome composite (1) (MSLC) includes liquid-filled liposomes (7) (LFLs) attached to the lipid (5) or surfactant (6). The presence of liquid-filled liposomes (7) stabilizes the surfactant-encapsulated or lipid-encapsulated gas-filled microsphere (3). One or more of the liquid-filled liposomes (7) will typically include liquid from the medium of suspension (2) (i.e., medium (2)). Preferably, each of the liquid-filled liposomes (7) will typically include liquid from the medium of suspension (2). The presence of liquid from the medium of suspension (2) in the liquid interior (10) (e.g., the interval volume) of the liquid-filled liposomes (7) provides for microsphere compositions that have densities which are close (e.g., within about 20%) to that of the medium of suspension (2), thereby minimizing microsphere flotation and/or separation.

The LFLs (7) can contain one or more drugs (e.g., therapeutic agents (8) and/or diagnostic agents (9)) in the liquid-filled internal volume. Because the LFLs (7) are attached to the surfactant-coated or lipid-coated gas-filled microsphere (3), the LFLs (7) can burst upon ultrasound stimulation of the internal gas thereby releasing the one or more drugs (e.g., therapeutic agents (8) and/or diagnostic agents (9)) in a diseased organ or tissue. The liquid-filled liposomes (7), however, have limited acoustic activity by themselves.

The LFLs (7) attach and stabilize the surfactant-encapsulated or lipid-encapsulated gas-filled microsphere (3). This provides for MSLCs (1) which have densities that are close (e.g., within about 20%) to that of the medium of suspension (2), thereby minimizing gas microsphere flotation and/or separation. This also provides for gas microsphere suspensions that are relatively uniform in size distribution (e.g., about 1 μm to about 5 μm) over a reasonable period of time after preparation (e.g., up to about 30 minutes).

The liquid-filled liposomes (7) typically occupy greater than about 50% of the microsphere surface area. The liquid-filled liposomes (7) are also typically attached to the adsorbed lipid (5) or surfactant (6) in an essentially continuous fashion. This orientation provides outstanding buoyancy properties for the MSLCs (1), which provides relatively stable suspensions with excellent and reproducible acoustic response properties when in dilute aqueous suspensions.

The size of the LFLs (7) is relatively important. The liquid-filled liposomes (7) should preferably have diameters that are less than about 10% of the diameter of the gas-filled microsphere (3) diameter. The range of greatest interest for most in vivo ultrasound imaging or drug delivery agents are MSLCs (1) that have an overall diameter between about 1 μm and about 5 μm, and are made from liquid-filled liposomes (7) of less than 100 nm in diameter. Larger liquid-filled liposomes (7) (e.g., greater than about 0.2 μm in diameter) create MSLCs (1) of overall diameter which exceed the diameter of the capillary vessels in the body. This would create a hazardous situation with respect to capillary plugging, as well as the consequent biological toxicity associated with blocking the microcirculation of blood to tissues. Therefore, it is highly preferred to utilize LFLs (7) of less than about 100 nm in diameter to create MSLCs (1) of the proper dimensions for safe use in living patients (e.g., mammals). Each of the liquid-filled liposomes (7) typically have a diameter of about 10 nm to about 200 nm. Preferably, each of the liquid-filled liposomes (7) will have a diameter of about 20 nm to about 100 nm. In addition, each of the liquid-filled liposomes (7) will typically have a diameter that is less than about 10% of the diameter of the gas-filled microsphere (3).

As illustrated in FIG. 2 and FIG. 3, one or more of the LFLs (7) may include one or more suitable drugs (e.g., therapeutic agents (9) and/or diagnostic agents (9)) in the liquid-filled internal volume. Each of the liquid-filled liposomes (7) may independently include one or more drugs (e.g., therapeutic agents (8) and/or diagnostic agents (9)) in the liquid interior (10) of the liquid-filled liposomes (7). The LFLs (7), when attached to the surfactant-coated or lipid-coated gas-filled microsphere (3) surface, can be burst upon ultrasound stimulation and release the one or more therapeutic drugs (e.g., therapeutic agents (8)) in a diseased organ or tissue in a localized and concentrated fashion. High energy ultrasound is generally capable of causing the gas-filled microsphere (3) to expand and contract rapidly, which eventually leads to gas bubble rupture. The ultrasound energy captured by the gas-filled microsphere (3) will cause the MSLC (1) to fragment and rupture, in turn, releasing the one or more drugs (e.g., therapeutic agents (8)) contained in the interior of the LFLs (7) attached to the surface of the MSLC (1).

Suitable classes of therapeutic agents (8) include, e.g., anticoagulants, thrombolytics, antineoplastic agents, and anti-inflammatory agents. Suitable specific therapeutic agents (8) are disclosed, e.g., in (PCT/US99/13682), and include, e.g., doxorubicin, cyclophosphamide, adriamycin, methotrexate, gemcitabine, navelbine, cisplatin, tissue plasminogen activator, integrelin, roxifiban, methotrexate and enbrel. In a preferred embodiment of the present invention for ultrasound stimulated drug release, the MSLCs (1) include both high affinity targeting moieties (11) and therapeutic drugs (e.g., therapeutic agents (8)) in the solution of the LFLs (7), in order to maximize the therapeutic index and the quantity of drug delivered per gas microbubble.

Suitable classes of diagnostic agents (9) include, e.g., X-ray contrast agents and MRI contrast agents. Suitable specific diagnostic agents (9) include, e.g., non-ionic iodinated X-ray contrast agents, ionic iodinated X-ray contrast agents, gadolinium containing MRI contrast agents, iron containing MRI contrast agents, and manganese containing MRI contrast agents.

The use of diagnostic agents (9) in the LFLs will allow both ultrasound image enhancement (e.g., back scatter) and X-ray or MRI image enhancement to be achieved with one MSLC composition.

For targeted delivery of one or more drugs (e.g., therapeutic agents (8) and/or diagnostic agents (9)) to a selected pathological condition, the LFLs (7) of the present invention can be derivatized with a high affinity, targeting moiety (11) that is covalently linked or adsorbed onto the surface (13) of the LFLs (7). As such, the liquid-filled liposomes (7) may typically have one or more suitable high affinity, targeting moieties (11) attached to the surface (13) of the liquid-filled liposomes (7). This provides LFLs (7) that are capable of providing ultrasound contrast enhancement to sites of pathology in vivo. This is accomplished by providing ligands on the LFLs (7) that have high affinity for receptors, enzymes, mRNA, or DNA which are overexpressed or altered in dysfunctional cells at sites of diseases. Alternatively, these targeting moieties (11) attached to the LFLs (7) can bind to normal tissue receptors for the selective imaging of normal tissues, in contrast to the absence of acoustic enhancement of the adjacent diseased tissue which lacks the receptor being targeted by the LFLs (7). One or more of the LFLs (7) may include, in the interior liquid medium (10), one or more suitable diagnostic agent (9) from the medium of suspension (2).

Suitable high affinity targeting moieties (11) which can be incorporated onto the surface (13) of the LFLs (7) for directing the MSLC (1) to specific sites of pathology have been disclosed previously. See, e.g., Unger (PCT/US96/09938), Allen (U.S. Pat. No. 5,620,689) and Quay (EP 0727225), which provide many examples of the biological targeting moieties that can be incorporated into surfactant (6) or lipid (5) components of directed ultrasound imaging agents or drug delivery compositions. Among these are tumor specific antibodies, receptor-specific peptides and peptidomimetics such as cell adhesion molecules and the like.

Suitable specific targeting moieties (11) include, e.g., 1,2-dipalmitoyl-sn-glycero -3-phosphoethanolamine-cyclo(Arg-Gly-Asp-D-Phe-Lys)-dodecanoate; DPPE-PEG₃₄₀₀-cyclo(Arg-Gly-Asp-D-Phe-Lys)-dodecanoate; 1-(1,2-Dipalmitoyl-sn-glycero-3-phosphoesphoethanolamino)-αω-di carbonyl PEG₃₄₀₀-2-{[7-(N-hydroxycarbamoyl)3S,6R,7S)-4-aza 6-(2-methylpropyl)-11-oxa-5-oxobicyclo[10.2.2]hexadeca-1(15),12(16),13-trien-3-yl]carbonylamino}-N-(3-aminopropyl)acetamide; and 1-(1,2-Dipalmitoyl-sn-glycero-3-phosphoethanolamino) -αω, dicarbonyl PEG₃₄₀₀-[7-(N-hydroxycarbamoyl)(3S,6R,7S)-4-aza-6-(2-methylpropyl)-11-oxa-5-oxobicyclo[10.2.2]hexadeca-1(15),12(16),13-trien-3-yl ]-N-{[4-(aminomethyl)phenyl]methyl}carboxamide.

The liquid-filled liposomes (7) used in the present invention are well known in the art. Among the preferred materials for making liposomes for use in the present invention are phospholipids which can be cationic, anionic or zwitterionic in nature, and may be used in admixtures. Many sources exist on the composition and preparation of liposomes. For example, see New (R. R. C. New, editor, Liposomes, a practical approach, Oxford University Press, Oxford, UK, 1990), Tyrrell (“New Aspects of Liposomes”, D. A. Tyrrell, T. D. Heath, C. M. Colley & B. E. Ryman, Biochimica & Biophysica Acta, 457 (1976), 259-302), Schneider (U.S. Pat. No. 4,224,179), Woodle (M C Woodle and D. Papahadjopoulos, Methods in Enzymology 171, 193, 1989). In particular, Papahajopoulos (U.S. Pat. No 4,235,871) has described methods for forming LFLs including therapeutic agents.

Control of MSLC Mean Size Distribution and Stability

The size and stability of the MSLCs can be controlled through several parameters, e.g., concentration of the lipid in solution; the diameter of the LFLs; the molecular weight of the polymer surfactant, e.g., polyethylene glycol (PEG), included in the composition; and the concentration of the polymer used.

1. The concentration of the phospholipids in solution

Varying the lipid concentration will control the size distribution and stability of the liquid filled liposome and, this in turn, will adjust the size of the MSLC which is formed and stabilized in suspension. The mean size of the stabilized MSLCs in suspension is directly proportional to the concentration and size of the initial LFLs. Since the number and size of the LFLs is dependent on the amount of lipid (e.g., phospholipid) available, the initial lipid concentration will directly effect the number and size of the MSLC distribution which is stabilized in suspension.

2. The diameter of the LFL Independent of the lipid (e.g., phospholipid) concentration, the size of the MSLCs in suspension can be varied by changing the LFL size through physical means. The LFL size can be varied by methods such as extrusion or ultrasonication, which are well-known in the science of liposomes (see, e.g., R. R. C. New, editor, Liposomes, a practical approach, Oxford University Press, Oxford, UK, 1990). As described previously, the variation of LFL size will result in varying MSLCs size distributions (i.e., smaller LFLs in the size range of less than about 100 nm will produce smaller MSLCs in the range of less than about 10 μm).

3. The molecular weight of surfactant used in the composition

The molecular weight of polymeric surfactant (ionic or non-ionic) in the preparation can be used to affect the mean diameter of the MSLCs formed. For example, a higher molecular weight of polyethylene glycol (PEG), either covalently bound to other components/lipids of the composition or added as free PEG in solution, can be used to stabilize larger sized gaseous microbubble-containing MSLCs once gas is introduced into the system. For example, by varying the molecular weight of the PEG from 500 to 10,000, the MSLC diameter can be adjusted.

4. Concentration of the polymer

The size of the MSLC can be controlled by changing the concentration of the polymer surfactant in the liposomal suspension. An increase in polymer concentration in the composition typically results in an increase in the mean size and/or concentration of the MSLCs in suspension.

Preparation of Gas Microsphere Liposome Composites

The gas microsphere liposome composites (MSLCs) described herein can be prepared by mixing a gas of low aqueous solubility with an aqueous solution containing a surfactant and liquid-filled liposomes in suspension. This can be accomplished by mechanical mixing, ultrasonication or high velocity injection of the gas into the liquid containing the surfactant and LFLs.

To form the initial LFLs, phospholipids can be suspended in a bulk aqueous solution, which can further include a surface active material, as well as non-aqueous components, such as glycerol or propylene glycol, or suspending aids such as polysaccharides, proteins or synthetic polymers, provided such components are parenterally acceptable (i.e., non-toxic). Methods for preparing the LFLs used in the current invention for preparation of MSLCs have been described previously by Woodle (M. C. Woodle and D. Papahadjopoulos, Methods in Enzymology 171, 193, 1989).

If biotargeting of the MSLC is desired, then the LFLs can have a high affinity targeting moiety covalently bound or adsorbed to the surface of the liquid-filled liposome. The targeting moieties can be adsorbed to the surface of the MSLC or, more preferably, covalently attached to the LFL as a phospholipid ester or attached to a PEG component of the MSLC (see Allen U.S. Pat. No. 5,620,689). In the case of MSLCs for ultrasound stimulated drug release, the LFLs can be prepared to include a therapeutic agent in the interior liquid volume of the liposomes by preparing the liposomes in a surfactant-containing, aqueous medium including the drug, followed by mixing or sonicating the medium with a suitable inert gas.

The control of the LFL diameter in the size range of less than about 100 nm is important for forming and stabilizing MSLCs of the desired size range (e.g., greater than about 0.5 μm and less than about 10 μm in diameter) for ultrasound imaging and ultrasound-stimulated drug release. Methods for controlling the size of LFLs have been described in the literature (see, e.g., R. R. C. New, editor, Liposomes, a practical approach, Oxford University Press, Oxford, UK, 1990, pp. 36-85). Microfluidization techniques for making LFLs of the desired size are particularly effective as described in Cook, et al., (U.S. Pat. No. 4,533,254).

Proof of Structure

To demonstrate the existence of this novel structure (termed gas microsphere liposome composite (MSLC)), the liposome system described in Example 1 was prepared and analyzed using four techniques, Optical Microscopy, Transmission Electron Microscopy, Fluorescence Probing and Soft X-ray Microscopy. These techniques provide information on the macrostructure (greater than about 1 μm in size), microstructure (10 nm to ˜1000 nm), and the microenvironment of the chemical system (at the molecular level).

Optical Microscopy

Optical Microscopy allows the determination of the size and shape of an object in the micron range. Therefore, a MSLC composition with a diameter in the range of about 1 to about 10 μm is visible using a 100×microscope, and will have a magnified size of about 1 to about 10 mm in diameter. Optical Microscopy was performed to show that the MSLCs are spherical in shape, and are present in the size range of about 1 to about 10 μm in diameter.

After preparation of the MSLC suspension (for example, as described in Example 1), about 0.5 mL is slowly withdrawn from the vial using a syringe (B-D 5 cc syringe and precision guide 22 1/2 G needle; 0.70 mm ×40 mm). The sample was placed on a Hanging Drop slide (18 mm diameter; 0.5 mm deep) and covered with a cover slide. Then a drop of microscope oil was placed on the cover slide. The sample was examined with an Olympus BHA-P Microscope equipped with 1×eyepiece and Oil Immersion Achromatic 100×Objective, which gave an overall magnification of 100×. The resulting picture showed spherical objects that range in size from less than 1 μm to greater than 10 μm. The gas filled MSLCs of greater than approximately 2 μm in diameter appear to be aggregates of the smaller-sized primary MSLC units.

Electron Microscopy

The presence of the LFLs on the surface of the MSLCs was demonstrated using transmission electron microscopy (TEM). Transmission electron microscopy uses an electron beam to illuminate a specimen. The electron beam is operated at high vacuum, and can magnify up to a 1,000,00×. Both the high vacuum and the electron beam can be damaging to the systems being studied. Therefore, in order for many samples to be examined, they must be thin, dry and usually contain a contrast stain.

One technique for examining liposome structures is negative staining. Negative staining enhances the image of a structure by surrounding or embedding the specimen in an electron dense material. The sample is examined under TEM using Phosphotungstic acid (PTA) as the stain, before mixing of the gas and aqueous system containing the surfactant-liposome mixture as well as after mixing to demonstrate formation of the MSLC.

For the surfactant-LFL system prior to mixing with the gas (the “unactivated” sample), six drops of the preactivated system were added to 1 ml of 0.3% PTA stain and shaken gently. The mixture was left to stand for 5 minutes undisturbed, and then one drop of the mixture was applied to the grid plate. The grid was air dried on a piece of filter paper for 30 minutes, before it was transferred to the grid carrying case for the TEM study.

For the MSLC sample (after mixing with the gas) one drop was added to 1 mL of 0.3% PTA stain and mixed gently. Then a drop of the solution was applied to the grid. The excess solution was removed by wicking and air-drying.

TEM pictures show that the composition, prior to mixing with a perfluorocarbon gas, contains liposomes of about 50 nm to about 100 nm. The TEM pictures of the post-mixing MSLC suspension (after mixing with a perfluorocarbon gas) show MSLCs of about 300 nm to about 1000 nm, which include a gas-filled microsphere void with a lipid or surfactant shell having liposome units of about 50 nm to about 100 nm along the surface.

Fluorescence Analysis

Fluorescent probe experiments were used to study the general chemical properties of a liposome system. A fluorescent probe is a fluorophore, typically pyrene, that localizes within a specific region of a liposome and responds to a photon of energy by producing a fluorescence emission. This emission can be used to determine the microenvironment (micropolarity) and localized concentration of the fluorophore in the system.

For this experiment, pyrene was injected into vials of control medium (solution without surfactant or liquid-filled liposomes), vials of surfactant and liquid-filled liposomes (prior to mixing with a perfluorocarbon gas) and into vials containing MSLCs in suspension (after high speed mixing of the composition with perfluoropropane) to compare the pyrene fluorescence spectra. The control medium that was used consisted of a mixture of 80% sodium chloride solution (9% NaCl), 10% propylene glycol and 10% glycerol. The MSLC suspension was prepared as described in Example 1.

The results of the study showed that the microenvironmental polarity of pyrene in the control medium was consistent with the pyrene being dissolved in a purely aqueous environment. The microenvironment polarity of the pyrene in the surfactant/liquid-filled liposome system (prior to gas mixing) was consistent with the pyrene being dissolved in the lipid membrane of the LFLs. Following high speed mechanical mixing of the perfluorocarbon gas with the surfactant/liquid-filled liposome system the local concentration of the pyrene was shown to increase in a manner consistent with the presence of a liposome aggregate system as such as the MSLC structure observed in the TEM experiment.

Soft X-ray Microscopy

Soft X-rays are X-rays with an energy of about 100 to about 1000 eV. These energies are well matched to K shell absorption edges of low Z atoms like carbon and oxygen, or L shell edges of atoms like calcium. The wavelength of these X-rays is in the 1 to 10 nm range, whereas those of visible light are 350-700 nm. This makes very high resolution imaging possible. Soft X-ray microscopy provides high resolution while avoiding sample destruction; the X-rays have negligible effects on the sample.

MSLC suspensions were studied using this Soft X-ray microscopy technique. The sample was prepared between two silicon nitride membranes. The membranes have a thickness of 100 nm and a size of 3 mm×3 mm in a 9 mm×9 mm silicon frame of 200 microns thickness. After mounting one membrane on each side of the wet cell, a syringe was used to put a very small droplet (less than about 5 μL, but not a defined volume) of the MSLC material on one of the membranes. For these experiments there is no dilution or pretreatment of the sample. Next, the two parts of the wet cell were placed together and tightened with screws. The layer thickness of the sample between the two membranes was checked with a visible light microscope. If the layer thickness was not appropriate the screws were adjusted to obtain the right thickness. A small droplet of water was placed into the reservoir slot of the wet cell to prevent evaporation of the sample. The reservoir slot was sealed with a small piece of tape and then the wet cell was mounted in the microscope.

The results from the Soft X-ray microscopy showed that the system after high speed mixing with perfluoropropane gas contained MSLCs of about 300 nm to about 500 nm having liquid-filled liposome units of about 50 nm to about 100 nm along essentially the entire boundary surface.

The MSLCs can be used as general purpose ultrasound contrast agents for diagnostic ultrasound use. They can also be modified to contain biological targeting moieties bound or adsorbed to the liquid filled liposomes on the surface of the MSLC to provide selective localization of the MSLCs in the body. Biologically-targeted MSLCs are useful for targeted contrast ultrasound imaging of specific disease processes. In addition, these biologically-targeted MSLCs can be used for localized delivery of drugs, which are encapsulated within the liquid-filled liposomes and are released upon exposure of the MSLCs to ultrasound energy in vivo.

The formulation may be administered intravenously or intraperitoneally by infusion or injection. Solutions of the formulation can be prepared in water, optionally mixed with a nontoxic surfactant. Dispersions can also be prepared in aqueous solutions containing glycerol, liquid polyethylene glycols, or other suitable parenteral diluents.

The pharmaceutical dosage forms suitable for injection or infusion can include sterile aqueous solutions or dispersions or sterile powders comprising the formulation which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle is a pharmaceutically acceptable diluent such as a mixture of water, ethanol, a polyol (for example, glycerol, propylene glycol, liquid polyethylene glycols), and the like. The proper fluidity can be maintained, for example, by the formation of liposomes, by the maintenance of the required particle size in the case of dispersions or by the use of surfactants. In many cases, it will be preferable to include isotonic agents, for example, sugars, buffers or sodium chloride. Prolonged suspension of the injectable compositions can be brought about by the use of agents such as gelatin, cellulose, polyvinyl pyrollidone or similar suspension aids.

Sterile injectable solutions are prepared by incorporating the required ingredients enumerated above, followed by filter sterilization. When employing sterile powders for the preparation of sterile injectable solutions, the preferred methods of preparation are vacuum drying and the freeze drying techniques, which yield a powder of the active ingredient(s) plus any additional desired ingredient present in the previously sterile-filtered solutions.

The microsphere liposome composites (MSLCs) are injected into a patient or human as a suspension containing approximately 10³ to 10⁹ microsphere liposome composites in a principally aqueous medium. After allowing sufficient time for the MSLCs to circulate throughout the body, an ultrasound imaging machine (such as is routinely used in clinical practice) is used to image or (with higher energies or repeated insonation pulses) disrupt the MSLCs to release a therapeutic drug at the site of disease or in an organ of interest, e.g., the heart, or in tumors, or at sites of inflammation.

The ability of a formulation of the invention to act as a contrast imaging agent can be determined using pharmacological models which are well known in the field. For example, see Villanueva et al. (Villanueva, F. S., Glasheen, W. P., Sklenar, J., Kaul, S. Circulation, 88, 596-604 (1993)).

The ability of a formulation of the invention to act as a therapeutic agent can be determined using pharmacological models which are well known in the field. For example, see Unger (PCT/US961/09938) (WO96/40285).

The invention will now be illustrated by the following non-limiting Examples.

Preparation of General Purpose Diagnostic MSLC Contrast Agent

### EXAMPLE 1

A saline glycerol solution (100 ml) was prepared including glycerol (10 ml) and NaCl (680±2 mg) in water (to a final volume of 100 ml). DPPC (dipalmitoyl phosphatidyl choline) (40.0 mg), MPEG500 DPPE (dipalmitoyl phosphatidyl ethanolamine) (30.0 mg), and DPPA (4.5 mg) were mixed with propylene glycol (10 ml) in a 100 ml volumetric flask, which was placed in a hot water bath (70° C.) and sonicated for 15 minutes until the solution cleared. The saline/glycerol solution was then added to bring the mixture to final volume of 100 ml, and the suspension was mixed well. The suspension (1.6 ml) was transferred into a 2 ml borosilicate glass vial. The headspace was purged with perfluoropropane gas, and the vial was stoppered and sealed. The stopper was West Gray V 50 lyo 13 mm, 4416/50 elastomeric formulation. The seal was a flip off aluminum seal. The vial containing the lipid suspension was shaken for 45 seconds using the IONOS Ionomix®. After shaking, the suspension became milky white.

Preparation of Biologically-Targeted Diagnostic MSLC Material

Examples 2 and 3 describe the synthesis of ultrasound contrast agents of the present invention comprising targeting moieties for tumor neovasculature that are αvβ3 antagonists.

### EXAMPLE 2

### Part A. Synthesis of 1,2-dipalmitoyl-sn-glycero-3-phosphoethanolamine-cyclo(Arg-Gly-Asp-D-Phe-Lys)-Dodecanoate Conjugate

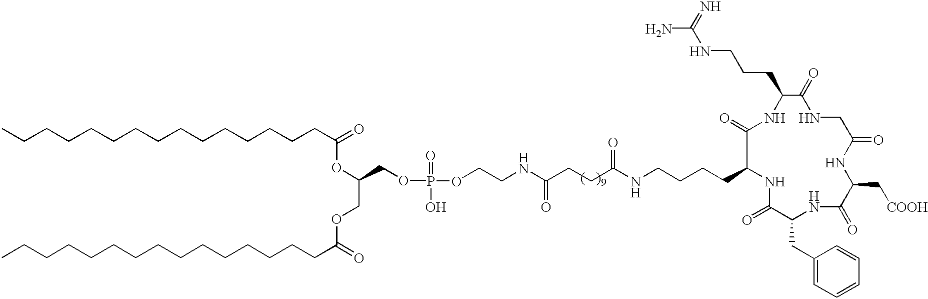

Disuccinimidyl dodecanoate (0.424 g, 1 mmol); 1,2-dipalmitoyl-sn-glycero-3-phosphoethanolamine (DPPE) (1.489 g, 1 mmol); and cyclo(Arg-Gly-Asp-D-Phe-Lys) TFA salt (0.831 g, 1 mmol) (see U.S. Ser. No. 09/281,474 for synthesis of this cyclic peptide targeting moiety, which method is herein incorporated by reference) are dissolved in chloroform (25 ml) while stirring (5 min). Sodium carbonate (1 mmol) and sodium sulfate (1 mmol) are added and the solution is stirred at room temperature under nitrogen (18 h). Chloroform is removed in vacuo and the title compound is purified from the crude product mixture by preparative HPLC or recrystallization.

### Part B. Preparation of Contrast Agent Composition

The 1,2-dipalmitoyl-sn-glycero-3-phosphoethanolamine-cyclo(Arg-Gly-Asp-D-Phe-Lys) -dodecanoate conjugate is mixed with three other lipids--1,2-dipalmitoyl-sn-glycero -3-phosphotidic acid; 1,2-dipalmitoyl-sn-glycero-3-phosphatidylcholine; and N-(methoxypolyethylene glycol 5000 carbamoyl)-1,2-dipalmitoyl-sn-glycero-3-phosphatidylethanolamine—in relative amounts of 2:4:54:40 by weight. An aqueous suspension containing this lipid mixture (1 mg/mL), sodium chloride (7 mg/mL), glycerin (0.1 mL/mL), and propylene glycol (0.1 mL/mL), at pH 6-7, is then prepared in a 2 cc glass vial. The air in the vial is evacuated and replaced with perfluoropropane, and the vial is sealed. The suspension is agitated in the sealed vial in a dental amalgamator for 30-45 sec. to form a milky white solution, which is suitable for use as an ultrasound contrast agent for imaging angiogenic vessels.

### EXAMPLE 3

### Part A. Preparation of ω-amino-PEG₃₄₀₀-cyclo(Arg-Gly-Asp-D-Phe-Lys):

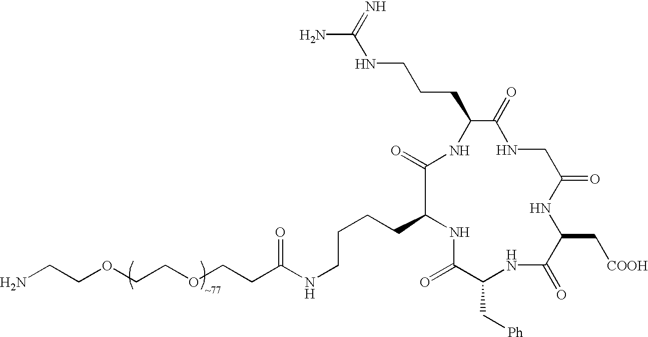

Triethylamine (3 mmol) is added to a solution of N-Boc-PEG₃₄₀₀-sucinimidyl ester (1 mmol) and cyclo(Arg-Gly-Asp-D-Phe-Lys) (1 mmol) in dimethylformamide (DMF) (25 mL). The reaction mixture is stirred under nitrogen at room temperature overnight, and the solvent is removed in vacuo. The crude product is dissolved in trifluoroacetic acid/dichloromethane (1:1 vol/vol) and stirred for 4 h. The volatiles are removed and the title compound is isolated as the TFA salt via trituration in diethyl ether.

### Part B. Preparation of DPPE-PEG₃₄₀₀-cyclo(Arg-Gly-Asp-D-Phe-Lys)-Dodecanoate Conjugate:

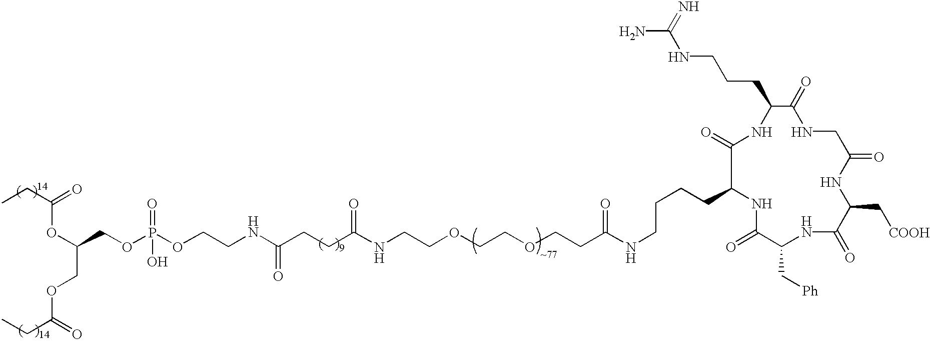

Disuccinimidyl dodecanoate (1 mmol), 1,2-dipalmitoyl-sn-glycero-3-phosphoethanolamine (DPPE) (1 mmol), and ω-amino-PEG₃₄₀₀-cyclo(Arg-Gly-Asp-D-Phe-Lys) TFA salt (1 mmol) are dissolved in chloroform (25 ml) while stirring for 5 min. Sodium carbonate (1 mmol) and sodium sulfate (1 mmol) are added and the solution is stirred at room temperature under nitrogen for 18 h. DMF is removed in vacuo and the title compound is purified from the crude product mixture by either preparative HPLC or recrystallization.

### Part C. Preparation of the Contrast Agent Composition:

The DPPE-PEG₃₄₀₀-cyclo(Arg-Gly-Asp-D-Phe-Lys)-Dodecanoate conjugate is mixed with three other lipids—1,2-dipalmitoyl-sn-glycero-3-phosphotidic acid; 1,2-dipalmitoyl sn-glycero-3-phosphatidylcholine; and N-(methoxypolyethylene glycol 5000 carbamoyl)-1,2-dipalmitoyl-sn-glycero-3-phosphatidylethanolamine—in relative amounts of 16:54:41 by weight. An aqueous suspension, containing this lipid mixture (1 mg/mL), sodium chloride (7 mg/mL), glycerin (0.1 mL/mL), and propylene glycol (0.1 mL/mL), at pH 6-7, is then prepared in a 2 cc glass vial. The air in the vial is evacuated and replaced with perfluoropropane, and the vial is sealed. The suspension is agitated in the sealed vial in a dental amalgamator for 30-45 sec. to form a milky white suspension, which is suitable for use as an ultrasound contrast agent.

The following examples, Examples 4 and 5, describe the synthesis of ultrasound contrast agents of the present invention comprised of targeting moieties for matrix metalloproteinase inhibitors. These materials are useful for targeting the MSLCs to the sites of extracellular matrix degradation, which are present in tumors, atherosclerotic plaques, and cardiac tissue degeneration in CHF (Congestive Heart Failure). These compositions are useful for localizing the acoustically active MSLCs to sites of disease for the selective ultrasound imaging of these pathologies. Alternatively, as described in Examples 8 and 9, compositions may be prepared with therapeutic agents in the interior of the LFLs attached to the MSLCs, which are useful for ultrasound stimulated drug release at a specific site of disease.

### EXAMPLE 4

### Synthesis of 1-(1,2-Dipalmitoyl-sn-glycero-3-phosphoethanolamino)-α,ω-dicarbonylPEG₃₄₀₀-2-{[7-(N-hydroxycarbamoyl)(3S,6R,7S)-4-aza-6-(2-methylpropyl)-11-oxa -5-oxobicyclo[10.2.2]hexadeca-1 (15),12(16),13-trien-3-yl]carbonylamino}-N-(3-aminopropyl)acetamide conjugate

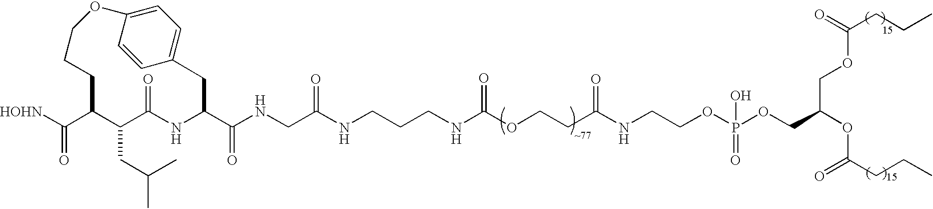

To a solution of succinimidyl ester DSPE-PEG-NHS ester (Shearwater Polymers, Huntsville, Ala.) (1 mmol) in chloroform (25 ml) is added 2-{[7-(N-hydroxycarbamoyl) (3S,6R,7S)-4-aza-6-(2-methylpropyl)-11-oxa-5-oxobicyclo[10.2.2]hexadeca-1(15),12(16),13-trien-3-yl]carbonylamino}-N-(3-aminopropyl)acetamide TFA salt (1 mmol) (see U.S. Ser. No. 60/182,627 for synthesis of this targeting moiety). Sodium carbonate (1 mmol) and sodium sulfate (1 mmol) are added and the solution stirred at room temperature under nitrogen for 18 h. The solvent is removed in vacuo and the title compound is purified from the crude product mixture by preparative HPLC.

Preparation of Contrast Agent Composition:

The 1-(1,2-Dipalmitoyl-sn-glycero-3-phosphoethanolamino)-α,ω-dicarbonylPEG₃₄₀₀-2-{[7-(N-hydroxycarbamoyl)(3S, 6R, 7S)-4-aza-6-(2-methylpropyl)-11-oxa -5-oxobicyclo[10.2.2]hexadeca-1(15), 12(16), 13-trien-3-yl]carbonylamino}-N-(3-aminopropyl)acetamide conjugate is mixed with three other phospholipids—1,2-dipalmitoyl-sn -glycero-3-phosphotidic acid; 1,2-dipalmitoyl-sn-glycero-3-phosphatidylcholine; and N-(methoxypolyethylene glycol 5000 carbamoyl)-1,2-dipalmitoyl-sn-glycero-3-phosphatidylethanolamine—in a ratio of 1:6:54:41 by weight. An aqueous suspension, containing this lipid mixture (1 mg/mL), sodium chloride (7 mg/mL), glycerin (0.1 mL/mL), and propylene glycol (0.1 mL/mL), at pH 6-7, is prepared in a 2 cc glass vial. The air in the vial is evacuated and replaced with perfluorobutane, and the vial is sealed. The suspension is agitated in the sealed vial in a dental amalgamator for 30-45 sec to form a milky white suspension of the MSLCs targeted to matrix metalloproteinases. The suspension is suitable for use as an ultrasound contrast agent.

### EXAMPLE 5

### Synthesis of 1-(1 2-Dipalmitoyl-sn-glycero-3-phosphoethanolamino)-α,ω-dicarbonylPEG₃₄₀₀-[7-(N-hy -oxobicyclo[10.2.2]hexadeca-1(15),12(16),13-trien-3-yl]-N-[4-(aminomethyl)phenyl]methyl}carboxamide conjugate:

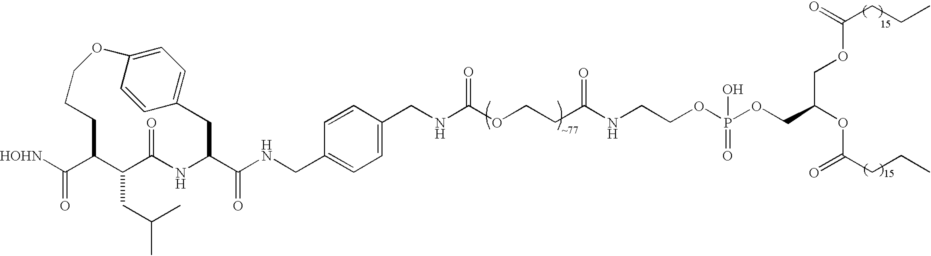

To a solution of succinimidyl ester DSPE-PEG-NHS ester (Shearwater Polymers, Huntsville, Ala.) (1 mmol) in chloroform (25 ml), is added [7-(N-hydroxycarbamoyl)(3S,6R,7S)-4-aza-6-(2-methylpropyl)-11-oxa-5-oxobicyclo[10.2.2]hexadeca-1(15),12(16),13-trien-3-yl]-N-{[4-(aminomethyl)phenyl]meth 60/182,627 for the synthesis of this MMP targeting moiety). Sodium carbonate (1 mmol) and sodium sulfate (1 mmol) are added and the solution is stirred at room temperature under nitrogen for 18 h. The solvent is removed in vacuo and the title compound is purified from the crude product mixture by preparative HPLC.

Preparation of Contrast Agent Composition

1-(1,2-Dipalmitoyl-sn-glycero-3-phosphoethanolamino)-α,ω-dicarbonylPEG₃₄₀₀-[7-(N-hydroxycarbamoyl)(3S,6R,7S)-4-aza-6-(2-methylpropyl)-11-oxa-5-oxobicyclo[10.2.2]hexadeca-1(15),12(16),13-trien-3-yl]-N-{[4-(aminomethyl)phenyl]methyl}carboxamide conjugate is mixed with three other phospholipids—1,2-dipalmitoyl-sn-glycero-3-phosphotidic acid; 1,2-dipalmitoyl-sn-glycero-3-phosphatidylcholine; and N-(methoxypolyethylene glycol 5000 carbamoyl)-1,2-dipalmitoyl-sn -glycero-3-phosphatidylethanolamine—in relative amounts of 1:6:54:41 by weight. An aqueous suspension (1.6 ml), containing this lipid mixture (1 mg/mL), sodium chloride (7 mg/mL), glycerin (0.1 mL/mL), and propylene glycol (0.1 mL/mL), at pH 6-7, is prepared in a 2 cc glass vial. The air in the vial is evacuated and replaced with perfluorobutane, and the vial is sealed. The suspension is agitated in the sealed vial in a dental amalgamator for 30-45 sec to form a milky white suspension of the MSLCs targeted to matrix metalloproteinases. The suspension is suitable for use as an ultrasound contrast agent.

Preparation of Biologically-Targeted Therapeutic MSLC Materials

### EXAMPLE 6

To the phospholipid contrast agent composition in Example 3 is added doxorubicin (100-200 mg/ml). One to two milliliters is transferred to a vial. The air in the vial is evacuated and replaced with perfluorobutane, and the vial is sealed. The vial is agitated in a dental amalgamator for 30-45 sec to form a milky white MSLC suspension for therapeutic use.

### EXAMPLE 7

To the phospholipid contrast agent composition in Example 4 is added cyclophosphamide (100-200 mg/ml). One to two milliliters is transferred to a vial. The air in the vial is evacuated and replaced with perfluorobutane, and the vial is sealed. The vial is agitated in a dental amalgamator for 30-45 sec to form a milky white MSLC suspension for therapeutic use.

### EXAMPLE 8

To the phospholipid contrast agent composition in Example 5 is added cyclophosphamide (100-200 mg/ml). One to two milliliters is transferred to a vial. The air in the vial is evacuated and replaced with perfluorobutane and the vial is sealed. The vial is agitated in a dental amalgamator for 30-45 sec to form a milky white MSLC suspension for ultrasonically-activated therapeutic use.

### EXAMPLE 9

To the phospholipid contrast agent composition in Example 5 is added tissue plasminogen activator (10-100 mg/ml). One to two milliliters is transferred to a vial. The air in the vial is evacuated and replaced with perfluorobutane and the vial is sealed. The vial is agitated in a dental amalgamator for 30-45 sec to form a milky white MSLC suspension for therapeutic use.

### EXAMPLE 10

Following injection into a living patient (e.g., mammal) and allowing sufficient time for the targeted MSLCs to localize at or near the site of disease, the diagnostic ultrasound scan may be acquired, or, in the case of therapeutic agent delivery, ultrasound energy of sufficient energy to disrupt the MSLCs and release the drug at the targeted site may be applied by either repeated pulsation or by application of very high power single pulses of ultrasound energy.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A formulation comprising a gas microsphere liposome composite suspended in a medium, wherein the gas microsphere liposome composite comprises:
a gas-filled microsphere;
at least one of a lipid and a surfactant adsorbed onto the surface of the gas-filled microsphere; and
liquid-filled liposomes attached to the lipid or surfactant.

2. The formulation of claim 1 wherein the gas of the gas-filled microsphere has a solubility of less than about 1.0% (v/v) in water at 25° C. and 1 atm.

3. The formulation of claim 1 wherein the gas-filled microsphere has an average diameter of about 0.1 μm to about 10 μm.

4. The formulation of claim 1 wherein the gas-filled microsphere has an average diameter of about 0.5 μm to about 10 μm.

5. The formulation of claim 1 wherein the gas-filled microsphere comprises at least one inert gas.

6. The formulation of claim 5 wherein the inert gas is a noble gas.

7. The formulation of claim 5 wherein the inert gas is a perfluoroether gas.

8. The formulation of claim 5 wherein the inert gas is a perfluorocarbon gas.

9. The formulation of claim 1 wherein the gas-filled microsphere has a lipid adsorbed onto the surface of the gas-filled microsphere.

10. The formulation of claim 1 wherein the gas-filled microsphere has a surfactant adsorbed onto the surface of the gas-filled microsphere.

11. The formulation of claim 1 wherein the lipid or surfactant forms a mono-molecular layer on the surface of the gas-filled microsphere.

12. The formulation of claim 1 wherein the lipid or surfactant forms a bi-molecular liposomal layer or multi-molecular liposomal layer on the surface of the gas-filled microsphere.

13. The formulation of claim 1 wherein the surfactant is a non-ionic surfactant, cationic surfactant, or anionic surfactant.

14. The formulation of claim 13 wherein the non-ionic surfactant comprises polyethylene glycol, polypropylene glycol, polyvinylpyrollidone, polyvinylalcohol, cellulose, gelatin, xanthan gum, pectin, or dextran.

15. The formulation of claim 13 wherein the cationic surfactant comprises a tetraalkyl ammonium, tetraalkyl phosphonium ion, or a suitable salt thereof.

16. The formulation of claim 15 wherein the cationic surfactant comprises a tetrahexyl ammonium, tetraoctyl ammonium, tetradecyl ammonium, tetrabutyl ammonium, tetrahexyl phosphonium, tetraoctyl phosphonium, tetrabutyl phosphonium, tetraphenyl phosphonium, or a suitable salt thereof.

17. The formulation of claim 13 wherein the anionic surfactant comprises an alkyl sulfonate, an alkyl carboxylate, or a suitable salt thereof.

18. The formulation of claim 17 wherein the anionic surfactant comprises dodecyl sulfate, palmityl sulfate, dodecyl carboxylate, palmityl carboxylate, or a suitable salt thereof.

19. The formulation of claim 1 wherein the lipid comprises a phospholipid, glycolipid, triglyceride or fatty acid.

20. The formulation of claim 19 wherein the phospholipid comprises dipalmitoylphosphatidyl choline, dimyristoylphosphatidyl choline, dilauryoylphosphatidyl choline, or dioleoylphosphatidyl choline.

21. The formulation of claim 1 wherein the liquid-filled liposomes are attached to the adsorbed lipid or surfactant in a continuous fashion.

22. The formulation of claim 1 wherein the liquid-filled liposomes occupy greater than about 50% of the outer surface of the gas-filled microsphere area.

23. The formulation of claim 1 wherein each of the liquid-filled liposomes independently has a diameter of about 10 nm to about 200 nm.

24. The formulation of claim 1 wherein each of the liquid-filled liposomes independently has a diameter of about 20 nm to about 100 nm.

25. The formulation of claim 1 wherein each of the liquid-filled liposomes independently has a diameter that is less than about 10% of the diameter of the gas-filled microsphere.

26. The formulation of claim 1 wherein each of the liquid-filled liposomes independently comprises a therapeutic agent or diagnostic agent in the interior of the liquid-filled liposomes.

27. The formulation of claim 26 wherein the therapeutic agent is an anticoagulant, thrombolytic, antineoplastic agent, or anti-inflammatory agent.

28. The formulation of claim 26 wherein the therapeutic agent comprises doxorubicin, cyclophosphamide, adriamycin, methotrexate, gemcitabine, navelbine, cisplatin, tissue plasminogen activator, integrelin, roxifiban, methotrexate or enbrel.

29. The formulation of claim 26 wherein the diagnostic agent comprises an X-ray contrast agent or an MRI contrast agent.

30. The formulation of claim 1 wherein each of the liquid-filled liposomes independently has high affinity, targeting moieties attached to the surface of the liquid-filled liposomes.

31. The formulation of claim 30 wherein the high affinity targeting moiety attached to the surface of the gas microsphere liposome composite comprises:
a ligand which binds to a receptor which is up-regulated in angiogenesis;
a ligand which binds to a receptor which is up-regulated in inflammation; or
a ligand which binds to a receptor which is up-regulated in atherosclerosis.

32. The formulation of claim 30 wherein the high affinity targeting moiety attached to the surface of the gas microsphere liposome composite comprises:
a ligand which binds to the integrons of αᵥβ₃,αᵥβ₅ or GpIIb/IIIa;
a ligand which binds to a matrix metalloproteinase; or
a ligand which binds to the LTB₄ receptor.

33. The formulation of claim 30 wherein the high affinity targeting moiety attached to the surface of the gas microsphere liposome composite comprises:
1,2-dipalmitoyl-sn-glycero-3-phosphoethanolamine-cyclo(Arg-Gly-Asp-D-Phe-Lys)-dodecanoate;
DPPE-PEG₃₄₀₀-cyclo(Arg-Gly-Asp-D-Phe-Lys)-dodecanoate;
1-(1,2-Dipalmitoyl-sn-glycero-3-phosphoethanolamino)-αω-odicarbonyl PEG₃₄₀₀-2-{[7-(N-hydroxycarbamoyl)(3S,6R,7S)-4-aza-6-(2-methylpropyl)-11-oxa-5-oxobicylo[10.2.2]hexadeca-1(15),12(16),13-trien-3-yl]carbonylamino}-N-(3-aminopropyl)acetamide; or
1-(1,2-Dipalmitoyl-sn-glycero-3-phosphoethanolamino)-(α,ω-dicarbonyl PEG₃₄₀₀-[7-(N-hydroxycarbamoyl)(3S,6R,7S)-4-aza-6-(2-methylpropyl)-11-oxa-5-oxo [10.2.2]hexadeca-1(15),12(16),13-trien-3-yl]-N-{[4-(aminomethyl)phenyl]methyl}carboxamide.

34. The formulation of claim 1 wherein each of the liquid-filled liposomes independently comprises liquid from the medium of suspension.

35. The formulation of claim 1 wherein the gas microsphere liposome composite has a mean diameter of about 0.1 μm to about 10 μm.

36. The formulation of claim 1 wherein the gas microsphere liposome composite has a mean diameter of about 0.2 μm to about 4 μm.

37. The formulation of claim 1 wherein the gas microsphere liposome composite exists as an aggregate of two or more gas microsphere liposome composites.

38. The formulation of claim 37 wherein the aggregate has a diameter of about 1 μm to about 100 μm.

39. The formulation of claim 1 wherein the gas microsphere liposome composite has a density of about 0.90 to about 1.10 of the density of the medium.

40. The formulation of claim 1 wherein the lipid or surfactant comprises a high affinity targeting moiety.

41. The formulation of claim 1 wherein the lipid or surfactant comprises a therapeutic agent.

42. The formulation of claim 41 wherein the therapeutic agent is doxorubicin, cyclophosphamide, adriamycin, methotrexate, gemcitabine, navelbine, cisplatin, tissue plasminogen activator, integrelin, roxifiban, methotrexate or enbrel.

43. The formulation of claim 1 wherein the medium comprises a diagnostic agent.

44. The formulation of claim 43 wherein the diagnostic agent is an X-ray or MRI contrast agent.

45. The formulation of claim 40 wherein the high affinity targeting moiety comprises:
1,2-dipalmitoyl-sn-glycero-3-phosphoethanolamine-cyclo(Arg-Gly-Asp-D-Phe-Lys)-dodecanoate;
DPPE-PEG₃₄₀₀-cyclo(Arg-Gly-Asp-D-Phe-Lys)-dodecanoate;
1-(1,2-Dipalmitoyl-sn-glycero-3-phosphoethanolamino)-α,ω-dicarbonyl PEG₃₄₀₀-2-{[7-(N-hydroxycarbamoyl)(3S,6R,7S)-4-aza-6-(2-methylpropyl)-11-oxa-5-oxobicyclo[10.2.2]hexadeca-1(15),12(16),13-trien-3-yl]carbonylamino}-N-(3-aminopropyl)acetamide; or
1-(1,2-Dipalmitoyl-sn-glycero-3-phosphoethanolamino)-α,ω-dicarbonyl PEG₃₄₀₀-[7-(N-hydroxycarbamoyl)(3S,6R,7S)-4-aza-6-(2-methylpropyl)-11-oxa-5-oxobicyclo[10.2.2]hexadeca-1(15),12(16),13-trien-3-yl]-N-{[4-(aminomethyl)phenyl]methyl}carboxamide.

46. A method of ultrasound imaging in a patient in need of such ultrasound imaging comprising:
administering to the patient an effective amount of a formulation of any one of claims 1-45;
allowing a sufficient period of time for the circulation of the gas microsphere composite to reach the targeted area; and
performing ultrasound imaging on the patient.

47. The method of claim 46 wherein the patient is a human.

48. The method of claim 46 wherein the effective amount of the formulation comprises about 10³ to about 10¹⁰ gas microsphere liposome composites.

49. The method of claim 46 wherein the sufficient period of time is about 5 minutes to about 2 hours.

50. The method of claim 46 wherein the sufficient period of time is about 5 to about 30 minutes.

51. A method of treating heart disease, inflammation, infection, cancer or thromboembolic disease in a patient in need of such treatment comprising:
administering to the patient an effective amount of a formulation of any one of claims 1-45, wherein one or more of the liquid-filled liposomes independently comprises a therapeutic agent;
allowing a sufficient period of time for the circulation of the gas microsphere composite to the targeted area; and
applying ultrasound energy to the region of pathology in the patient sufficient to cause the therapeutic agent to be released from the microsphere liposome composite at the region of pathology.

52. The method of claim 51 wherein the patient is a human.

53. The method of claim 51 wherein each of the liquid-filled liposomes independently comprises a therapeutic agent.

54. The method of claim 51 wherein the effective amount of the formulation comprises about 10³ to about 10¹⁰ gas microsphere liposome composites.

55. A method for preparing a formulation of any one of claims 1-45 comprising:
contacting a suspension of liposomes in a aqueous solution comprising at least one lipid or one surfactant; and
mixing the suspension with a gas that has a solubility of less than about 1.0% (v/v) in water at 25° C. and 1 atm sufficient to provide the formulation.

56. The method of claim 55 wherein the mixing is accomplished by mechanical agitation.

57. The method of claim 55 wherein the mixing is accomplished by ultrasonification.

58. The method of claim 55 wherein the mixing is accomplished by high speed injection of the gas into the aqueous liposome suspension.

59. A method for preparing a formulation of any one of claims 1-45 comprising:
contacting a suspension of liposomes in a aqueous solution comprising at least one therapeutic agent and at least one surfactant; and
mixing the aqueous liposome suspension with a gas that has a solubility of less than about 1.0% (v/v) in water at 25° C. and 1 atm sufficient to provide the formulation.

60. The method of claim 59 wherein the mixing is accomplished by mechanical agitation.

61. The method of claim 59 wherein the mixing is accomplished by ultrasonification.

62. The method of claim 59 wherein the mixing is accomplished by high speed injection of the gas into the aqueous liposome suspension.

63. A kit for the preparation of a formulation of any one of claims 1-45 comprising:
a container comprising an aqueous solution wherein the aqueous solution comprises at least one surfactant and liquid-filled liposomes; and
a means for introducing a gas that has a solubility of less than about 1.0% (v/v) in water at 25° C. and 1 atm into the aqueous solution.

64. The kit of claim 63 wherein the container comprises a head space.

65. The kit of claim 63 wherein the head space comprises at least one inert gas having a solubility of less than about 1.0%(v/v) in water at 25° C. and 1 atm pressure.

66. The kit of claim 63 wherein the inert gas is a perfluorcarbon gas.

67. The kit of claim 63 wherein the inert gas is a perfluoroether gas.

68. The kit of claim 63 wherein the inert gas is a noble gas.

69. A formulation comprising a gas microsphere liposome composite suspended in a medium, wherein the gas microsphere liposome composite comprises:
a gas-filled microsphere;
at least one of a lipid and a surfactant adsorbed onto the surface of the gas-filled microsphere; and
liquid-filled liposomes attached to the lipid or surfactant;
for use in medical therapy or diagnosis.

70. The use of a formulation comprising a gas microsphere liposome composite suspended in a medium, wherein the gas microsphere liposome composite comprises:
a gas-filled microsphere;
at least one of a lipid and a surfactant adsorbed onto the surface of the gas-filled microsphere; and
liquid-filled liposomes attached to the lipid or surfactant;
for the manufacture of a medicament for treating heart disease, inflammation, infection, cancer or thromboembolic disease in a patient in need of such treatment.

71. The use of a formulation comprising a gas microsphere liposome composite suspended in a medium, wherein the gas microsphere liposome composite comprises:
a gas-filled microsphere;
at least one of a lipid and a surfactant adsorbed onto the surface of the gas-filled microsphere; and
liquid-filled liposomes attached to the lipid or surfactant;
for the manufacture of a medicament for diagnostic imaging in a patient in need of such diagnostic imaging.

72. The use of a formulation comprising a gas microsphere liposome composite suspended in a medium, wherein the gas microsphere liposome composite comprises:
a gas-filled microsphere;
at least one of a lipid and a surfactant adsorbed onto the surface of the gas-filled microsphere; and
liquid-filled liposomes attached to the lipid or surfactant;
for the manufacture of a medicament for ultrasound imaging in a patient in need of such ultrasound imaging.
